(19) United States
(12) Patent Application Publication
(10) Pub. No.: US 2006/0275859 A1
(43) Pub. Date: Dec. 7, 2006

(54) Enzyme sensor including a water-containing spacer layer

(76) Inventor: Thomas Kjaer, Ballerup (DK)

Correspondence Address: MORGAN LEWIS & BOCKIUS LLP, 1111 PENNSYLVANIA AVENUE NW, WASHINGTON, DC, 20004, US

(21) Appl. No.: 11/434,252

(22) Filed: May 16, 2006

Related U.S. Application Data
(60) Provisional application No. 60/754,322, filed on Dec. 29, 2005.

(30) Foreign Application Priority Data
May 17, 2005 (DK) PA 2005/00718
Jul. 18, 2005 (DK) PA 2005/01064

(51) Int. Cl. C12Q 1/26; C12M 1/34

(52) U.S. Cl. 435/25; 435/287.1; 205/777.5

(57) ABSTRACT

The present disclosure relates to an amperometric enzyme sensor including a water-containing spacer layer in contact with an electrode. The sensor is useful determining the presence or amount of biological analytes, e.g. glucose, lactate, creatine, creatinine, etc.

### RELATED APPLICATIONS

The present application claims the benefit of Danish application PA 2005/00718 (filed May 17, 2005), Danish application PA 2005/01064 (filed Jul. 18, 2005) and U.S. provisional application 60/754,322 (filed Dec. 29, 2005), each of which is incorporated herein in its entirety by reference.

### FIELD OF THE INVENTION

The present invention relates to an amperometric enzyme sensor including a water-containing spacer layer in contact with an electrode.

### BACKGROUND OF THE INVENTION

Enzyme sensors are sensors where a chemical species to be measured (an analyte) undergoes an enzyme catalysed reaction in the sensor before detection. The reaction between the analyte and the enzyme (for which the analyte is a substrate), or a cascade of enzymes, yields a secondary species which concentration (under ideal conditions) is proportional with or identical to the concentration of the analyte. The concentration of the secondary species is then detected by a transducer, e.g., by means of an electrode.

The enzyme of an enzyme sensor is typically included in a sensor membrane suitable for contacting a fluid sample. Most typically, the enzyme is included in a separate enzyme layer of the sensor membrane, which is separated from the fluid sample by means of a cover membrane. Hence, the analyte is contacted with the enzyme after diffusion through the cover membrane of the sensor, the enzyme/analyte reaction then takes place, and the secondary species then diffuses to the detector part of the sensor, e.g., an electrode, to yield a response related to the analyte concentration.

On the other hand, the enzyme layer is most often separated from the electrode by an interference limiting layer which allows the secondary species to diffuse there through. Traditional enzymatic H₂O₂ detecting sensors normally comprise a cover membrane for the enzyme layer (i.e., a diffusion limiting layer), an enzyme layer, an intermediate layer (i.e., an interference limiting layer) and a metal anode. Such systems generally perform satisfactorily when they are used for analytes which are present in high concentrations (e.g., as lactate and glucose in blood samples). However, if the system is applied to analytes which are present in very low concentrations (e.g., creatinine, creatine or other analytes with a detection limit in the range of about 1 to about 20 μM), it has been observed that fluid samples without the analyte can cause a significant false signal on the electrode. The false signals may correspond to a signal from about −25 μM to about 25 μM analyte, and they stem from differences (other than the analyte, which is zero) in the composition of the various liquids brought into contact with the enzyme sensor, e.g., blood samples, cleaning liquids, wetting liquids, calibration liquids, etc. The way of action is probably, a combination of two different reasons:

First, non-ionic species diffuse more rapidly across the interference limiting layer than ionic species. Therefore, bicarbonate/CO₂ present in the fluid sample but not in the rinse solution will cause the pH below the interference limiting layer to drop. The same effect is seen with imidazole/H-imidazole being present in most rinse solutions but not in the samples. A drop in pH will cause a drop in the zero current that stems from oxidation of water.

Second, the concentration of ionic species at the anode surface changes as a function of the different samples. Such changes will lead to changes in the ionic composition on the electrode, thereby leading to a current known as a non-faradaic current. The total amount of electric charge being transported as non-faradaic current will only depend on the difference in ionic composition; however, the time constant of the diffusion can be changed.

False signals are particularly problematic for differential measurement, e.g., upon determination of the concentration of an analyte such as creatinine in a blood sample.

Thus, there is a need for enzyme sensors wherein false signals caused by the use of liquids of different compositions are reduced or even eliminated.

US Published Application 2004/0011671 A1 discloses a device and method for determining analyte levels, in particular to implantable devices for monitoring glucose levels in a biological fluid.

WO 90/05910 A1 discloses a wholly micro-fabricated biosensor comprising an analyte attenuation layer.

### SUMMARY OF THE INVENTION

It has been found that the above problem can be alleviated by introducing a water-containing spacer layer between the anode and the interference limiting layer. More specifically, it has been found that the present invention has rendered it possible to achieve a higher degree of accuracy and reliability for enzyme sensors, namely by providing an enzyme sensor for determining the concentration of an analyte in a fluid sample, said sensor comprising an electrode, a water-containing spacer layer in contact with said electrode, at least one intermediate layer, the innermost of said at least one intermediate layer being in contact with said spacer layer, and at least one enzyme layer, the innermost of said at least one enzyme layer being in contact with the outermost of said at least one intermediate layer.

### BRIEF DESCRIPTION OF THE DRAWINGS

The following figures are merely exemplary embodiments of the invention and are in no way intended to limit the scope of the disclosure.

FIG. 1 illustrates a conventional enzyme sensor comprising an electrode and a membrane.

FIG. 2 illustrates the membrane of the sensor of FIG. 1.

FIG. 3 illustrates an exemplary planar, thick-film sensor construction.

FIG. 4 illustrates the effect of the introduction of a water-containing spacer layer on false signals for a dual sensor system when contacted with various liquids.

FIG. 5 illustrates the sensitivity of a sensor covered by a water-containing spacer layer and an interference eliminating layer to H₂O₂.

### DETAILED DESCRIPTION OF THE INVENTION

As described herein, the present invention relates to an amperometric enzyme sensor for determining the concentration of an analyte in a fluid sample.

As described herein, the term “enzyme sensor” is generally intended to encompass electrochemical sensors comprising an enzyme (or an enzyme cascade) which is capable of converting an analyte of interest into a secondary species. The analyte of interest is a possible constituent of the fluid sample, i.e., the enzyme sensor is typically used for determining the concentration of the analyte in the fluid sample. The “analyte” is sometimes referred to as an “enzyme substrate”, or simply a “substrate”.

The sensors of the invention are typically multi-use sensors. A multi-use sensor is to be understood as a sensor which is used for more than one measurement and, thus, is exposed to more than one volume of sample and may possibly have intermittent contact with calibration liquids, cleaning liquids, etc. Such sensors are typically used for a longer period of time.

The fluid sample can in principle be any liquid which is compatible with the sensor, and in particular the cover membrane. In an exemplary embodiment, the fluid sample is an aqueous liquid. Fluid samples include physiological fluids, such as urine, saliva, interstitial fluids, spinal fluid and blood. Blood includes whole blood samples, diluted blood samples, blood fractions, pre-reacted blood samples, etc. The sensors are particularly well-suited for whole blood samples.

The sensor of the invention may be of the conventional type or of the planar type, e.g., a thick-film sensor or a thin-film sensor. The enzyme membranes of such sensors are often referred to as layered membranes.

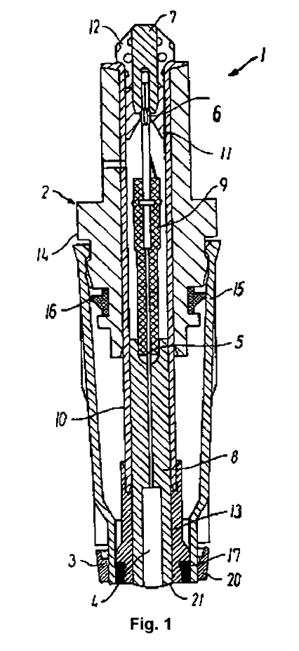

In the case of enzyme sensors of the conventional type, a membrane, e.g., a multi-layered membrane comprising, for example a spacer layer, an intermediate layer, an enzyme layer, and a cover membrane, is typically assembled as a discrete object which is then arranged in conjunction with (i.e., generally mounted on the tip of) an electrode. See, e.g., FIG. 1. Methods for the construction of such a multi-layered membrane are well-known in the art. See, e.g., WO 98/21356. Enzyme sensors of the conventional type may include track-etched membranes as well as solvent-cast membranes.

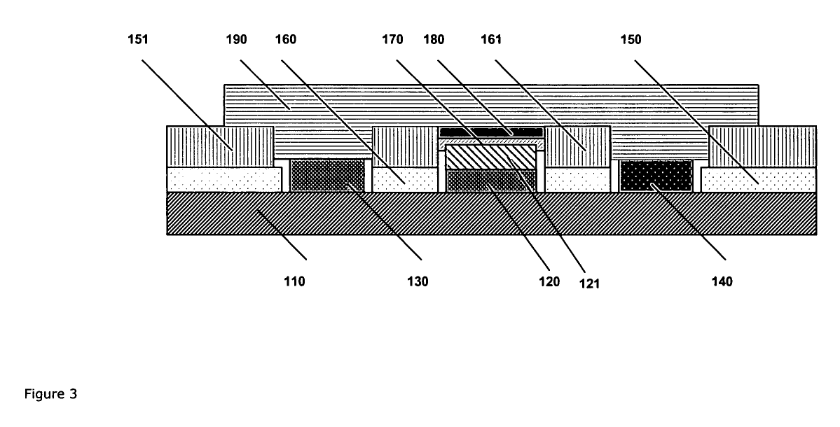

In case of enzyme sensors of the planar type, e.g., thick-film sensors and thin-film sensors, the electrode and the enzyme membrane comprising the water-containing spacer layer are arranged by depositing materials (typically sequentially and individually) corresponding to the electrode, the spacer layer, the intermediate layers, the enzyme layer, and the cover layer, respectively, onto a solid, dielectric substrate, e.g., a ceramic or wafer material. An example of a planar sensor construction is illustrated in FIG. 3. Methods for the construction of planar type sensors, e.g., thick-film sensors and thin-film sensors, are well-known in the art. See, e.g., WO 01/90733, WO 01/65247 and WO 90/05910. The materials corresponding to the layers of such sensor membranes are most often deposited by solvent-casting.

The enzyme sensors of the invention comprise an electrode, a water-containing spacer layer in contact with said electrode, at least one intermediate layer, the innermost of said at least one intermediate layer being in contact with said spacer layer, and at least one enzyme layer, the innermost of said at least one enzyme layer being in contact with the outermost of said at least one intermediate layer.

The spacer layer of the enzyme sensor is generally described in its ready-to-use form, i.e., the form where the water-containing spacer layer contains a substantial amount of water, and wherein the enzyme sensor is capable of measuring an analyte of a fluid sample. The enzyme sensor is, however, typically stored and delivered to the end-user in dry form, i.e., in a form where the spacer layer is substantially dry. Thus, the end-user will have to wet the membrane of the enzyme sensor with an aqueous liquid so that spacer layer, which is capable of absorbing a substantial amount of water, is converted into the water-containing spacer layer. Other layers may also be able to absorb a substantial amount of water.

In a conventional enzyme sensor construction, the wetting is typically conducted by means of the internal liquid of the enzyme sensor (see e.g., FIG. 1). Planar sensors are typically wetted by, for example, specific wetting liquids, cleaning liquids or calibration liquids, etc.

The principal parts of the enzyme sensor are (i) an electrode, (ii) a water-containing spacer layer which is in contact with said electrode, (iii) at least one intermediate layer, where the innermost of said at least one intermediate layer is in contact with said spacer layer, and (iv) at least one enzyme layer, where the innermost of said at least one enzyme layer is in contact with the outermost of said at least one intermediate layer. The spacer layer, the at least one intermediate layer, the at least one enzyme layer and optionally a cover layer in conjunction forms an enzyme membrane of the enzyme sensor. The individual parts will be described in details in the following.

Water-Containing Spacer Layer

The enzyme sensors of the invention include a water-containing spacer layer separating the electrode and at least one intermediate layer.

The term “water-containing spacer layer” is intended to mean a layer which, when the sensor is in use, provides a buffering effect in the sense that pH instability at the electrode surface is reduced.

It is believed that the water in the water-containing spacer layer buffers the changes in ionic composition experienced by the anode, thus extending the non-faradaic current over a longer time interval and resulting in a current with smaller amplitude. Diffusion is a very rapid process at small distances (typically less than 1 s for O₂ diffusion over 50 μm); therefore, the spacer layer does not function in isolation, but only in combination with a diffusion resistance (e.g., the interference limiting layer), so that the system functions like a capacitor in series with a resistor. As such, the interference limiting layer should be rather impermeable to ions, or else the spacer layer should be very thick.

The high water content of the spacer layer warrants that the analyte (e.g., H₂O₂) can easily and rapidly diffuse across the layer. Computer modelling of the sensor signals with and without the water-containing spacer layer supports these findings. For example, an amplitude loss of less than 5% and an increase in time constant from 13.0 s to 13.2 s was observed for the sensor system described below.

Suitable examples of materials forming the porous polymeric matrix of the water-containing spacer layer for conventional sensors (track-etched or solvent-cast) include, but are not limited to, polyesters, such as polyethylene terephthalate (PETP), glycol-modified polyethylene terephthalate (PETG), and glycol-modified polycyclohexylenedimethylene terephthalate (PCTG), polycarbonates, celluloses (e.g., regenerated, acetate, triacetates, acetate butyrates), polyolefins and derivatives thereof, fluorinated hydrocarbon polymers and copolymers (e.g., polychlorotrifluoroethylene, polyvinylidene fluoride, polytetrafluoroethylene, polyethylene chlorotrifluoroethylene, polyethylene tetrafluoroethylene, fluorinated ethylene-propylene copolymer), polyimides (e.g., Kapton), polystyrene, poly(meth)acrylates, polyvinyl chloride and derivatives thereof (including copolymers such as vinyl chloride-co-(meth)acrylate-type copolymer), polyamides, polyurethanes, polysulphones, polyethersulphones, polyphenylene sulphide, silicones, and copolymers of organosiloxane-polycarbonate (e.g., those disclosed in U.S. Pat. No. 3,189,662), in particular polyethylene terephthalate (PETP), polyvinyl chloride, and polycarbonate. In one embodiment, the material comprising the space layer for such sensors is polyethylene terephthalate (PETP). Preferably, such spacer layers are track-etched.

Suitable examples of materials forming the porous polymeric matrix of the water-containing spacer layer for planar sensors, e.g., thick-film sensors (solvent-cast), include, but are not limited to, polymers selected from hydrophilic polyurethanes, hydrophilic poly(meth)acrylates, poly(vinyl pyrrolidone), polyurethanes, Nafion™-polymers, electropolymerised polymers (e.g., polythiophenes, 1,3-diaminobenzenes, phenols), and SPEES-PES (polyaryl-ethersulphone/polyethersulphone copolymer). Alternatively, the material forming the porous polymeric matrix may be selected from the same materials as defined immediately above for the spacer layer of a conventional sensor mixed with a porosity forming compound (e.g., detergents or water-soluble hydrophilic polymers), in particular polyvinyl chloride, and polycarbonate, mixed with such porosity forming compounds.

In the present context, the term “water-containing” as used in connection with the spacer layer, is intended to mean that the porous polymeric matrix comprises a substantial amount of water, e.g., an amount of at least about 6% based on the weight of the porous polymeric matrix. The water content may be even higher, e.g., at least about 8%, such as at least about 10% or at least about 20%, or at least about 25% or at least about 40% or at least about 50%, or higher. For solvent-cast planar sensors, the total degree of swelling (i.e., water-uptake) should be carefully considered, because an excessively high water-uptake may be detrimental to the structural integrity of the enzyme membrane. Thus, for planar sensors, the water content should preferably not exceed about 200%, such as about 150%.

In an exemplary embodiment of the invention, the water-containing spacer layer further comprises one or more components selected from a buffer, a cation-exchange material and an electrolyte salt (e.g., an electrolyte polymer) in order to further reduce the effect of bicarbonate (HCO₃⁻) in the fluid samples and other liquids.

The spacer layer may have a porosity in the range of about 0.0005 to about 2% (vol/vol) for track-etched materials and in the range of about 1 to about 90% for solvent-cast materials.

For conventional creatinine/creatine and urea sensors with a track-etched spacer layer, the porosity may preferably be in the range of 0.05-0.1%, such as 0.2-0.25%. For conventional lactate sensors with a track-etched spacer layer, the porosity may preferably be in the range of 0.0005-0.015%, such as 0.003-0.004%. For conventional glucose sensors with a track-etched spacer layer, the porosity may preferably be in the range of 0.001-0.05%, such as 0.01-0.02%. The porosity for track-etched membranes is determined as: porosity (%)=π×(pore diameter/2)²×(pore density)×100%. The average pore size of the spacer layer may be in the range of about 0.05 to about 250 nm, such as about 1 to about 150 nm, or about 10 to about 110 nm, and the pore density may be in the range of about 40,000 to about 40,000,000 pores per cm².

The porosity for solvent-cast spacer layers may most easily be determined as the volume occupied by water when the membrane is wetted with water. The porosity of solvent-cast spacer layers may be in the range of about 1 to about 90%, such as about 3 to about 85%. The difference of at least one order of magnitude between the porosity of track-etched membranes and solvent-cast membranes can be explained by the fact that only the “effective” pores of the track-etched membranes are taken into account, whereas all pores and cavities are included in the determination of porosity for the solvent-cast membranes.

The weight ratio between the water and the solid matter of the spacer layer may be in the range of from about 10:1 to about 1:10, e.g., from about 8:1 to about 1:5, or from about 10:1 to about 1:2.

The water-containing spacer layer typically has a thickness in the range of about 0.2 to about 20 μm, such as about 0.5 to about 15 μm. For planar sensors, the thickness is typically in the range of about 0.2 to about 10 μm, such as about 0.5 to about 5 μm. For conventional sensors, the thickness is typically in the range of about 1 to about 20 μm, such as about 2 to about 15 μm.

The water-containing spacer layer may either be in the form of a solvent-cast layer or in the form of a track-etched membrane. It is often desirable, in particular for spacer layers for planar sensors, e.g., thick-film sensors, to mix the above-mentioned polymeric materials with a porosity forming compound (e.g., detergents, water-soluble hydrophilic polymers, etc.) in order to obtain a suitable porosity.

In the case of conventional sensors, it is preferred to use track-etched membranes because it is believed to be important that the pores are oriented substantially perpendicular relative to the electrode surface (see, e.g., FIG. 1) so that the secondary species to be detected at the electrode is directed more accurately to the electrode surface. This results in a reduced diffusion from border areas thus providing a faster sensor response. Further, with this construction, the radial electrical contact is minimized and thereby external electrical noise is reduced.

In one embodiment, the sensor is of the conventional type and the water-containing spacer layer is a track-etched polyethylene terephthalate material. In another embodiment, the sensor is of the planar type and the water-containing spacer layer is a solvent-cast layer of hydrophilic polyurethane or hydrophilic poly(meth)acrylate, preferably mixed with a porosity forming compound, e.g., selected from detergents, water-soluble hydrophilic polymers, etc.

For the various embodiments defined above, it is preferred that the combination of the water-containing spacer layer and at least one intermediate layer separating the electrode from the at least one enzyme layer is capable of limiting the diffusion of compounds such as paracetamol, ascorbic acid and uric acid in such a manner that the signal is reduced by at least about 90%, such as at least about 95%, for an initial period of about 15 seconds.

Intermediate Layer

In one embodiment, the enzyme layer is not in direct contact with the spacer layer, and so the enzyme sensor may include at least one intermediate layer that preferably functions as an interference limiting layer.

In another embodiment, the at least one intermediate layer is selected from, for example, cellulose acetate (CA), Nafion™, hard PVC, Baytron™, electropolymerised polymers (e.g., polythiophenes, 1,3-diaminobenzenes, phenols), and SPEES-PES (polyaryl-ethersulphone/polyethersulphone copolymer). In one embodiment, the at least one intermediate layer is an interference limiting cellulose acetate (CA) layer.

Combined Spacer Layer and Intermediate Layer

In an alternative variant, the water-containing spacer layer and the at least one intermediate layer are combined into a heterogeneous layer of materials of the type described for the intermediate layer and materials of the type described for the spacer layer. The layer is formed in such a manner that the material of the spacer layer type is dispersed in a continuous phase of the material of the at least one intermediate layer type. The selections with respect to materials and properties are as described above for the spacer layer and the at least one intermediate layer.

Electrode

The electrode of the enzyme sensor is selected with due respect to the reaction products of the analyte and the enzyme(s) (e.g., an enzyme cascade as for the creatinine sensor). Typically, the electrode is prepared from a precious metal, e.g., gold, palladium, platinum, rhodium, indium or iridium, preferably gold or platinum, or mixtures hereof. Other suitable electron-conductive materials include MnO₂, Prussian blue, graphite, iron, nickel and stainless steel.

In some instances, it is preferred to further include additional electrodes, e.g., an internal reference electrode and/or a counter electrode, adjacent to the mandatory electrode. See, e.g., FIG. 3.

Enzyme Layer

The enzyme layer (or layers) of the enzyme sensor plays an important role in that the one or more enzymes facilitate the conversion of the analyte to a secondary species which can be detected at the electrode surface. In some embodiments, a single enzyme is used (e.g., glucose oxidase, lactate oxidase, urease), whereas a plurality of enzymes (e.g., creatinase or sarcosine oxidase) may be used to facilitate a cascade of reactions leading to a species which can be detected.

The enzyme(s) may either be deposited as such, or in a direct or indirect immobilised form, e.g., embedded or mixed in a polymer, or cross-linked, or immobilised to an underlying layer or to the cover layer so as to reduce or eliminate migration. In some embodiments, a plurality of enzymes may be arranged in separate layers. The enzyme layer may also be held in place by a ring or gasket so as to avoid use of excessive amounts of enzyme and so as to ensure that a well-determined amount of enzyme is placed in a well-defined region of the sensor membrane.

The at least one enzyme layer may comprise at least one enzyme including, but not limited to, carbohydrate oxidase, glucose oxidase, galactose oxidase, glycolate oxidase, aldose oxidase, pyranose oxidase, lactate oxidase, alpha-hydroxy acid oxidase, sarcosine oxidase, alcohol oxidase, glycerol oxidase, amine oxidase, amino acid oxidase, cholesterol oxidase, urease, bilirubin oxidase, laccase, peroxidase, glucose dehydrogenase, lactate dehydrogenase, glutamate dehydrogenase, P-450, superoxide dismutase, catalase, creatininase, creatinase, and related co-enzymes.

For detection of creatine, the enzyme layer preferably comprises creatinase and sarcosine oxidase. For detection of creatinine, the enzyme layer preferably comprises creatininase, creatinase and sarcosine oxidase. For detection of glucose, the enzyme layer preferably comprises glucose oxidase. For detection of lactate, the enzyme layer preferably comprises lactate oxidase. For detection of urea, the enzyme layer preferably comprises urease.

Cover Membrane

The enzyme sensor may also include a cover membrane of at least one porous polymeric material so as to ensure that a limited, well-defined, representative amount of the analyte is allowed to diffuse into the enzyme layer, i.e., under controlled conditions. Such an analyte-limited conversion is a prerequisite for obtaining a substantially linear relation between the sensor response and the analyte concentration within a reasonable range.

In an exemplary embodiment, the enzyme sensors of the invention comprise a diffusion limiting layer in the form of a cover membrane which is adapted to separate the enzyme layer from the fluid sample. The cover membrane is preferably a porous membrane which limits the diffusion of the analyte into the enzyme layer so that the capacity of the immobilised enzyme for conversion of the analyte is not exceeded, and so that sufficient oxygen (O₂) for the enzymatic conversion of the analyte is present in the enzyme layer. The principle of diffusion limiting layers is described in, for example, Danish Patent No. 170103. Thus, virtually any known cover membrane may be useful in connection with enzyme sensors of the invention.

In an exemplary embodiment, the diffusion of the analyte through the cover membrane is invariable over time and from sample to sample, so that an identical analyte concentration for two separate samples gives rise to a well-defined sensor response. In another embodiment, the cover membrane is capable of allowing fast diffusion of a small amount of the analyte across the membrane, thus facilitating an even dispersion of the analyte in the enzyme layer so that an enzyme of the enzyme layer immediately converts the analyte to a secondary species, giving rise to a rapid sensor response. Such an almost simultaneous conversion of the analyte results in an improved linearity. Moreover, it is desirable that macromolecules, e.g., proteins and enzymes, are substantially prevented from migrating across the cover membrane. It has been noted that proteases present in, e.g., a cleaning solution or a blood sample will have adverse effects on the enzyme layer if such proteases are allowed to migrate into and through the cover membrane.

On the other hand, it is also important that the cover membrane is capable of providing a high retention of the secondary species (e.g., H₂O₂ and O₂) within the sensor so that the response derived from the secondary species is not biased by substantial amounts of those species diffusing out through the pores of the cover membrane, and so that a sufficient amount of O₂ is retained within the enzyme layer in order to maintain analyte limited conversion. These features are particularly relevant to consider if an intermediate layer having a diffusion limiting effect is arranged between the enzyme layer and the electrode.

The at least one porous polymeric material may be selected from a fairly wide range of materials. Illustrative examples include polyesters, such as polyethylene terephthalate (PETP), glycol-modified polyethylene terephthalate (PETG), and glycol-modified polycyclohexylenedimethylene terephthalate (PCTG), polycarbonates, celluloses (regenerated, acetate, triacetates, acetate butyrates), polyolefins and derivatives thereof, fluorinated hydrocarbon polymers and copolymers (e.g., polychlorotrifluoroethylene, polyvinylidene fluoride, polytetrafluoroethylene, polyethylene chlorotrifluoroethylene, polyethylene tetrafluoroethylene, fluorinated ethylene-propylene copolymer), polyimides (e.g., Kapton), polystyrene, poly(meth)acrylates, polyvinyl chloride and derivatives thereof (including copolymers such as vinyl chloride-co-(meth)acrylate-type copolymer), polyamides, polyurethanes, polysulphones, polyethersulphones, polyphenylene sulphide, silicones, and copolymers of organosiloxane-polycarbonate (e.g., those disclosed in U.S. Pat. No. 3,189,662).

In an aspect of the invention, the at least one porous polymeric material is selected from polyethylene terephthalate (PETP), polyvinyl chloride, and polycarbonate.

In one embodiment, the porous polymeric material is polyethylene terephthalate (PETP).

In another embodiment, the porous polymeric material is polyvinyl chloride (PVC).

Typically, the at least one porous polymeric material does not comprise a hydrophilic polyurethane, because it is believed that such a material will provide an excessive level of H₂O₂ diffusion, especially if an intermediate layer with a diffusion limiting effect is included.

In one embodiment, a cover membrane possessing several of these favourable characteristics is achieved in that the outer surface and pore mouths of at least one face of the at least one porous polymeric material are covered by a hydrophilic polymer, preferably selected from hydrophilic polyurethanes and hydrophilic poly(meth)acrylates.

The cover membrane (and thereby also the at least one porous polymeric material) comprises two faces, one face that is proximal to the enzyme layer and one face that is distal to the enzyme layer, the latter furthermore facing the fluid sample when the enzyme sensor is in use. As described above, at least one face of the at least one porous polymeric material is covered by a hydrophilic polymer.

In an aspect of the invention, at least the face that is distal to the enzyme layer is covered by a hydrophilic polymer. This aspect provides advantages with respect to reduction or even elimination of blood bias and blood drift, and extends the lifetime of the sensor.

In another aspect, at least the face that is proximal to the enzyme layer is covered by a hydrophilic polymer. It is expected that the problems relating to varying sensitivity, lack of linearity, analyte distribution in the enzyme layer and reduced lifetime can be reduced or eliminated in this manner. If the at least one porous polymeric material is properly selected, e.g., by choosing a porous polymeric material that itself has a suitable blood compatibility, problems relating to blood bias and blood drift may at least in part be reduced, even in the absence of hydrophilic polymer covering the outer surface and pore mouths of the face of the at least one porous polymeric material distal to the enzyme layer.

In a preferred embodiment, both faces are covered by a hydrophilic polymer. This arrangement provides advantages with respect to reduction or even elimination of blood bias and blood drift, analyte distribution in the enzyme layer, improvement of sensitivity and linearity, extends the lifetime of the sensor, limits the enzyme migration and improves linearity.

The expression “outer surface and pore mouths” refers to each of the two faces of the at least one porous polymeric material which represent a surface interrupted by pore mouths (pore openings).

In the present context, the expression “covered by” refers to the fact that not only the surface of the at least one porous polymeric material, but also the pore mouths are covered by the hydrophilic polymer (e.g., selected from hydrophilic polyurethanes and hydrophilic poly(meth)acrylates).

The expression “a hydrophilic polymer” as used herein is intended to refer to a single hydrophilic polymer as well as a mixture of two or more hydrophilic polymers. It should be understood that the hydrophilic polymer(s) described above may be mixed with up to 30% of other non-hydrophilic polymers. In one preferred embodiment, however, the coating on the at least one porous polymeric material only comprises a hydrophilic polymer selected from hydrophilic polyurethanes and hydrophilic poly(meth)acrylates.

The use of a hydrophilic polymer selected from, for example, hydrophilic polyurethanes and hydrophilic poly(meth)acrylates to cover the outer surface and pore mouths of the at least one porous polymeric material also renders it possible to tailor the diffusion properties to obtain the desired diffusion restriction to make the enzyme membrane suitable for different sample analyte concentration ranges, and depending on the different porosities of the porous polymeric material prior to coating it.

For planar sensors, a coating of the hydrophilic polymer (e.g., selected from, for example, hydrophilic polyurethanes and hydrophilic poly(meth)acrylates) is typically obtained by dispensing, spraying, screen-printing, etc. a solution of the hydrophilic polymer onto the surface (and pore mouths) of the at least one porous polymeric material. In an aspect of the invention, the outer surface and pore mouths of at least one face of the at least one porous polymeric material most distal to the enzyme layer are covered by the hydrophilic polymer. An alternative embodiment, i.e., the one where the enzyme layer is coated with a hydrophilic polymer before establishing the at least one porous polymeric material (and optionally coating the porous polymeric material with the same or another hydrophilic polymer) is also envisaged, just as the embodiment where both faces are coated.

For conventional sensors, a coating on the at least one porous polymeric material may be obtained by dispensing, spraying, screen-printing, etc. a solution of the hydrophilic polymer onto the surface (and pore mounts) of the at least one porous polymeric material (either both faces or just one face thereof), or the at least one porous polymeric material may be submerged into a solution of the hydrophilic polymer, etc.

Hence, in particular for the conventional sensors with track-etched porous materials, both faces of the at least one porous polymeric material may be covered by the hydrophilic polymer. The fact that the face of the at least one porous polymeric material proximal to the enzyme layer may also be covered by the hydrophilic polymer is believed to provide particular advantages, in particular for track-etched porous polymeric materials, because a relatively large distance between individual pores gives rise to a non-linear response in the absence of a coating of a hydrophilic polymer. In this situation, the analyte (enzyme substrate) will have to diffuse to non-occupied enzyme molecules within the enzyme layer, and a longer diffusion distance results in a non-simultaneous conversion. In contrast, a coating of the hydrophilic polymer on the face of the at least one porous polymer material proximal to the enzyme layer will facilitate diffusion of the analyte within the layer and provide the analyte more evenly to the enzyme layer, whereby a higher or more linear response is obtained. Thus, with a coating of the hydrophilic polymer on the face of the at least one porous polymer material proximal to the enzyme layer the analyte will only have to travel a short distance in the denser enzyme layer until it reaches an available enzyme molecule.

In some aspects of the invention, not only the outer surface and the pore mouths of the at least one porous polymeric material is covered by the hydrophilic polymer, but the hydrophilic polymer has also at least partly penetrated the pores of the porous polymeric material from at least one face thereof.

In these particular embodiments, the at least one porous polymeric material of the cover membrane layer is said to be at least partly impregnated with the hydrophilic polymer. In the present context, the term “impregnated” is intended to mean that the hydrophilic polymer covers the outer surface and pore mouths of both faces of the at least one porous polymeric material and also has penetrated the pores of the porous polymeric material.

The term “at least partly impregnated” is intended to mean that the hydrophilic polymer covers the outer surface and pore mouths of at least one face of said at least one porous polymeric material and also has at least partly penetrated the pores of the porous polymeric material originating from said at least one face.

In another aspect of the invention, the hydrophilic polymer is substantially insoluble in water upon use of the sensor. However, the hydrophilic polymer is preferably not cross-linked when applied to the cover membrane in order to cover the same, and preferably no subsequent cross-linking takes place. Instead, the hydrophilic and water-insoluble properties of the hydrophilic polymer are obtained by a suitable combination of hydrophilic segments and hydrophobic segments/moieties of the hydrophilic polymer. This arrangement provides a much simplified procedure of manufacture because a cross-linking step can be completely omitted.

The term “insoluble in water” is intended to refer to a polymer that does not substantially dissolve in water upon storage of a cover membrane covered by the hydrophilic polymer for 24 hours at 25° C. in an aqueous solution.

In an embodiment of the invention, the at least one porous polymeric material has a porosity of in the range of about 0.002 to about 30% (vol/vol).

The desired porosity of the polymeric material depends to a certain extent on the desired upper limit of the detection range. A very high upper limit of the detection range will require a fairly low porosity so as to obtain a broad linear range, such that the cover membrane should present a fairly high diffusion resistance for the analyte. When expressed as a mathematical product of the porosity (% (vol/vol)) and the upper limit of the linear detection range (mM of analyte), the value is preferably in the range of about 0.01 to about 50 [% (vol/vol)·mM], such as about 0.05 to about 10 [% (vol/vol)·mM] or about 0.1 to about 2 [% (vol/vol)·mM].

In an exemplary embodiment, the average pore size of the porous polymeric material is in the range of about 0.05 to about 250 nm, such as about 1 to about 150 nm, or about 10 to about 110 nm.

In one embodiment, in particular where the sensor is of the conventional type, the porous polymeric material is a track-etched with a pore density in the range of about 40,000 to about 40,000,000 pores per cm².

For creatinine/creatine and urea sensors with track-etched cover membranes, the porosity may be in the range of about 0.05 to about 0.1%, such as about 0.2 to about 0.25%. For lactate sensors with track-etched cover membranes, the porosity may be in the range of about 0.0005 to about 0.015%, such as about 0.003 to about 0.004%. For glucose sensors with track-etched cover membranes, the porosity may be in the range of about 0.001 to about 0.05%, such as about 0.01 to about 0.02%. The porosity for track-etched membranes is determined as: porosity (%)=π×(pore diameter/2)²×(pore density)×100%.

The porosity for solvent-cast membranes may more easily be determined as the volume occupied by water when the membrane is wetted with water. The porosity of solvent-cast membranes is typically in the range of about 1 to about 40%, such as about 3 to about 30%. The difference of at least one order of magnitude between the porosity of track-etched membranes and solvent-cast membranes can be explained by the fact that only the “effective” pores (i.e., through-going pores) of the track-etched membranes are taken into account, whereas all pores and cavities are included in the determination of porosity for the solvent-cast membranes.

In one embodiment, the hydrophilic polymer is a hydrophilic polyurethane.

Polyurethanes are the most widely used biomedical polymers for blood-contacting surfaces, e.g., for implants and medical devices. Polyurethane elastomers are multiphase block copolymers which consist of alternating blocks of hard and soft segments. Hydrophobic hard segments are formed in the reaction of aliphatic, cycloaliphatic or aromatic diisocyanates with diols, diamine or water. The soft hydrophilic or relative hydrophilic segments are composed of low-molecular weight hydroxy-terminated polyethers, polyesters or aliphatic polyolefins. The hydrophilic polyols are used as chain extenders or can alternatively be incorporated in the prepolymer. Chemical incompatibility between the hard and soft segments and between the hydrophobic and hydrophilic segments leads to phase segregation in polyurethanes. The hard segment domains, which are interconnected with secondary bonds and dispersed in the soft segment matrix, act as physical cross-links reinforcing the whole system. The soft matrix can be tailored in respect to hydrophilicity by using mixtures of polyethers or polyesters with different hydrophilicities. For very hydrophilic polyurethanes, polyethylene glycol is often used, and the tailoring of their hydrophilic properties can be accomplished with the higher polyalkyl ethers, e.g., polypropylene glycol and polybutylene glycol. In this way polyurethanes can be produced as hydrophilic, hydrophobic, hydrophilic/hydrophobic, hard and stiff or soft and elastic, hydrolytically stable or deliberately degradable. Because of their hard and soft segmented structure the polyurethanes are mechanically strong, tear resistant and exhibit good flex life. These properties make the polyurethanes suitable as hydrophilic coatings for sensor membranes. The coatings have high water absorption due to the content of hydrophilic segments and good in-use stability. Due to their pseudo cross-linked segmented structure, the coatings are also insoluble in water.

The hydrophilic polyurethane may be selected from polyurethanes having hydrophilic segments included therein, e.g., segments of polyethylene glycol, polypropylene oxide, etc. Such hydrophilic polyurethane may be prepared from polyalkylene glycols (polyalkylene oxides) having terminal hydroxy or amino groups thereby forming linear polymer chains by reaction with di-isocyanates. Examples of such hydrophilic polyurethanes are those disclosed in U.S. Pat. Nos. 4,789,720; 4,798,876; and 5,563,233. Other suitable examples include polyurethanes modified with hydrophilic groups, e.g., aliphatic polyethers. See, e.g., U.S. Pat. No. 6,200,772 B1.

The hydrophilic segments are typically derived from polyethylene glycols, amino-group terminated polyethylene glycols, polypropylene glycols, amino-group terminated polypropylene glycols, polyethylene oxide, polypropylene oxide, and polyethylene imines, in particular polyethylene glycols.

In some embodiments, the hydrophilic polyurethane is selected from aliphatic polyether urethanes, aliphatic polyether urethaneureas, cycloaliphatic polyether urethanes, cycloaliphatic polyether urethaneureas, aromatic polyether urethanes, aromatic polyether urethaneureas, aliphatic polyester urethanes, aliphatic polyester urethaneureas, cycloaliphatic polyester urethanes, cycloaliphatic polyester urethaneureas, aromatic polyester urethanes, and aromatic polyester urethaneureas. Aliphatic polyether urethanes or cycloaliphatic polyether urethanes (e.g., cyclohexyl polyether urethanes) are preferred as a membrane coating, where linear or cyclic aliphatic diisocyanates are used. Isocyanates of natural origin (e.g., lysine-diisocyanate) are also suitable. Cyclohexyl polyether urethanes are believed to provide good biocompatibility to the membrane and to suppress or even eliminate fouling of the membrane.

In an aspect of the invention, the hydrophilic polyurethanes comprise backbone segments of polyethylene glycol, i.e., —(CH₂—CH₂—O—)ₙ—, in particular in a weight ratio of polyethylene glycol segments of at least about 5% (w/w), such as at least about 7% (w/w) or at least about 10% (w/w). A significant content of polyethylene glycol segments is expected to provide proper hydrophilic characteristics and to improve blood compatibility.

Suitable examples of preferred hydrophilic polyurethanes are those disclosed in U.S. Pat. No. 5,322,063 which is hereby incorporated by reference.

In an aspect of the invention, the hydrophilic polyurethane comprises backbone segments of polysaccharides (e.g., alginate, carrageenanes, pectin and dextranes), poly(HEMA), partly hydrolysed polyvinyl acetate (PVA) or cellulose derivatives (e.g., hydroxyethyl methyl cellulose, and carboxymethyl cellulose), in an exemplary weight ratio of the respective polysaccharide, polyvinyl acetate or cellulose derivative segments of at least about 5% (w/w), such as at least about 7% (w/w) or at least about 10% (w/w).

Examples of suitable commercially available hydrophilic polyurethanes include Hydromed D4 (water content when wetted: 50% (w/w)) and Hydromed D640 (water content when wetted: 93% (w/w)). Both polyurethanes are tradenames of Cardiotech International Inc., Wilminton, Mass., USA.

The Hydromed D4 and D640 products comprise a central polybutyleneoxide segment and polyalkyleneoxide terminal groups. The polyalkylenoxide groups may either be polyethyleneoxides or polyethyleneoxide-polypropyleneoxide-polyethyleneoxide. In both instances, the polyethyleneoxide segments are preferably longer than the length of the polybutyleneoxide and polypropyleneoxide segments. This appears to facilitate sufficient hydrophilicity and water-absorption as well as a suitable blood compatibility.

In another embodiment, the hydrophilic polymer is a hydrophilic poly(meth)acrylate.

Examples of hydrophilic poly(methacarylates) include acrylic copolymers with first monomer units consisting of an acrylic ester having a poly(ethylene oxide) substituent as part of the alcohol moiety of the ester, and a one or more second monomer units selected from methacrylates and acrylates. The poly(ethylene oxide) substituent of the first monomer units typically has an average molecular weight of about 200 to about 2000, e.g., about 500 to about 1500. Examples of such first monomers are methoxy poly(ethylene oxide)methacrylates, methoxy poly(ethylene oxide)acrylates, etc. Examples of the second monomer units include methyl methacrylate, ethyl acrylate, butyl methacrylate, etc.

Preferred hydrophilic poly(meth)acrylates include the acrylic copolymers disclosed in WO 93/15651 A1 which is hereby incorporated by reference in its entirety.

In an exemplary embodiment, a combination of monomers is methoxy poly(ethylene oxide)methacrylates, ethyl acrylate and methyl methacrylate.

In another exemplary embodiment, hydrophilic poly(meth)acrylates include those having segments or side chains of poly(vinyl pyrrolidone) (PVP).

In an aspect of the invention, the hydrophilicity of the hydrophilic polymer is such that the water content, when wetted, is in the range of about 5 to about 100% (w/w), or about 10 to about 95% (w/w), such as about 25 to about 95% (w/w), or about 45 to about 95% (w/w). The water content is typically a function of the type and content of hydrophilic polymers in the sense that a higher content of hydrophilic segments gives rise to a higher water-content (when wetted). Porosity may also play a role with respect to the preferred range for the water content, i.e. for membranes having small pores, an exemplary range for the water content may be about 5 to about 80% (w/w), such as about 8 to about 40% (w/w), such as about 10 to about 30% (w/w).

The properties of the cover membrane with respect to diffusion, diffusion rate and ability to exclude particularly large molecules are important to the functioning of the sensor.

Also to be considered is the ability of the cover membrane to allow diffusion of glucose and on the other hand to limit the diffusion of H₂O₂. Thus, in one embodiment, the diffusion rate of H₂O₂ through the cover membrane relative to the diffusion rate of glucose through the cover membrane is in the range of about 3 to about 20, such as about 3 to about 15, or such as 3 to about 10. Diffusion rate is determined as described in the “Experimentals” section. The relative diffusion rates for the cover membrane are superior to the rates of a typical, known polyurethane cover membrane.

In an exemplary embodiment, the apparent diffusion coefficient for glucose through the cover membrane is in the range of about 0.1 to about 5.0×10⁻⁹, such as about 0.3 to about 1.5×10⁻⁹, or such as about 0.5 to about 1.1×10⁻⁹ for a glucose sensor. In an exemplary embodiment for a corresponding lactate sensor, the apparent diffusion coefficient for lactate through the cover membrane is in the range of about 0.5 to about 5×10⁻¹⁰, such as about 1.2 to about 3.2×10⁻¹⁰. Apparent diffusion coefficients are measured as described in the “Experimentals” section.

Of further relevance is the ability of the cover membrane to exclude “large” molecules (e.g., peptides, proteins and enzymes such as the enzymes of the enzyme layer (e.g., glucose oxidase and lactate oxidase)), while at the same time allowing diffusion of the relevant analyte, e.g., lactate, glucose, creatine, creatinine, etc. Such analytes typically have a molecular weight of up to about 200 whereas peptides, proteins and enzymes may have molecular weights of from about 300 for small peptides to several thousands or more for proteins, e.g., about 30,000 for glucose oxidase. The cover membrane layer (in wet form) may have a thickness of in the range of about 5 to about 40 μm, such as about 6 to about 30 μm, or such as about 10 to about 17 μm, for conventional sensors. For thick-film sensors, the cover membrane layer (in wet form) may have a thickness of in the range of about 1 to about 20 μm, such as about 2 to about 10 μm, or such as about 3 to about 5 μm.

In an exemplary embodiment, the hydrophilic polymer layer of the cover membrane in dry form has a thickness of in the range of about 0.1 to about 5 μm, such as about 0.25 to about 3 μm, or such as about 0.5 to about 1 μm, particularly for thick-film sensors.

In an exemplary embodiment, the hydrophilic polymer layer of the cover membrane in dry form has a thickness of in the range of about 100 to about 2000%, such as about 100 to about 1000%, or such as about 200 to about 500%, of the average size of the pores of the polymeric material.

In view of the preferred water-absorption properties, the ratio between the thickness of the cover membrane in wet form and the thickness of the cover membrane in dry form may be in the range of about 2:1 to about 1:1.

In an exemplary embodiment, the ratio between the thickness of the hydrophilic polymer layer of the cover membrane in wet form and the thickness of the hydrophilic polymer layer of the cover membrane in dry form is in the range of about 100:1 to about 1:1, or such as about 80:1 to about 2:1.

In some embodiments, in particular for conventional sensors, the ratio between the thickness of the hydrophilic polymer layer of the cover membrane in wet form and the thickness of the hydrophilic polymer layer of the cover membrane in dry form may be in the range of about 20:1 to about 1.5:1, such as in the range of about 10:1 to about 2:1. In some other embodiments, in particular for conventional sensors, e.g., with track-etched membranes, the ratio between the thickness of the hydrophilic polymer layer of the cover membrane in wet form and the thickness of the hydrophilic polymer layer of the cover membrane in dry form is preferably in the range of about 80:1 to about 10:1, such as in the range of about 50:1 to about 30:1.

For planar sensors, e.g., with solvent-cast membranes, the ratio between the thickness of the hydrophilic polymer layer of the cover membrane in wet form and the thickness of the hydrophilic polymer layer of the cover membrane in dry form may be in the range of about 10:1 to about 2:1, such as in the range of about 6:1 to about 3:1.

In other embodiments, the weight ratio between the porous polymeric material and the hydrophilic polymer (non-wetted) is in the range of about 100:1 to about 1:1, e.g., about 80:1 to about 10:1, or about 50:1 to about 30:1.

In one embodiment of the invention, the cover membrane is the outermost layer of the enzyme sensor.

Several advantages have been identified by using, for example, hydrophilic polyurethane to cover the outer surface (and pore mouths) of at least one face of the at least one porous polymeric material. For one, the polyurethane in itself has small pores which effectively block the pores of the porous polymeric material for penetration/migration of enzymes/proteins, while still allowing for the diffusion of smaller hydrophilic and hydrophobic molecules. Furthermore, the hydrophilic polyurethane is normally not soluble in water, although the polyurethane is swellable and is capable of holding substantial amounts of water. As such, leaching and degeneration of the polyurethane coating will be substantially absent during the lifetime of the sensor. The same also applies to, for example, hydrophilic poly(meth)acrylates.

One exemplary embodiment relates to an amperometric enzyme sensor for determining the concentration of creatine in a fluid sample, said sensor comprising a metal electrode (e.g., platinum electrode), a water-containing spacer layer, e.g., of polyethylene terephthalate (PETP), in particular of a track-etched PETP material, in contact with said metal electrode, an interference limiting layer, e.g., of a cellulose acetate (CA), in contact with said spacer layer, an enzyme layer comprising, for example, sarcosine oxidase and creatinase in contact with said cellulose acetate layer, and a cover membrane layer for said enzyme layer, wherein said cover membrane layer comprises a porous polyethylene terephthalate material, and wherein the outer surface and pore mouths of at least one face of the at least one porous polymeric material are covered by a hydrophilic polyurethane comprising backbone segments of polyethylene glycol in a weight ratio of polyethylene glycol segments of at least about 5% (w/w) and/or have a water content when wetted of at least about 25% (w/w).

Another exemplary embodiment relates to an amperometric enzyme sensor for determining the concentration of creatinine in a fluid sample, said sensor comprising a metal electrode (e.g., platinum electrode), a water-containing spacer layer, e.g., of polyethylene terephthalate (PETP), in particular of a track-etched PETP material, in contact with said metal electrode, an interference limiting layer, e.g., of a cellulose acetate (CA), in contact with said spacer layer, an enzyme layer comprising, for example, sarcosine oxidase, creatininase and creatinase in contact with said cellulose acetate layer, and a cover membrane layer for said enzyme layer, wherein said cover membrane layer comprises a porous polyethylene terephthalate material, and wherein the outer surface and pore mouths of at least one face of the at least one porous polymeric material are covered by a hydrophilic polyurethane comprising backbone segments of polyethylene glycol in a weight ratio of polyethylene glycol segments of at least about 5% (w/w) and/or have a water content when wetted of at least about 25% (w/w).

Use of Enzyme Sensors

The enzyme sensors of the invention may be exposed to a wetting or calibration fluid before its first use, normally until the signal is stabilized.

Measurements of, e.g., creatinine, creatine, glucose, lactate, etc. in samples of physiological fluids may take place in various automated or semi-automated analysers, many of which employ multiple sensors to measure multiple parameters. One example is a clinical analyser, particularly a blood analyser. The fluid sample is introduced manually or automatically into a flow system of the analyser or into a flow system of a cassette for introduction into the analyser. Sensors for one or more parameters of the physiological sample may thus be exposed to the fluid sample introduced into the flow system.

Hence, the present invention also provides an apparatus for determining the concentration of an analyte in a fluid sample, said apparatus comprising one or more enzyme sensors as described herein.

Furthermore, the present invention also provides a method of determining the concentration of an analyte in fluid sample, said method comprising the steps of contacting the fluid sample with an enzyme sensor as described herein, and conducting at least one measurement involving the electrode of the enzyme sensor.

The sensors are normally exposed to the sample and other fluids, e.g., wetting fluids, cleaning fluids, calibration fluids, etc. that are conducted to and from the sensor.

The above description is not intended to limit the claimed invention in any manner. Furthermore, the discussed combination of features might not be absolutely necessary for the inventive solution. In addition, the disclosures of all patents or published applications cited herein are incorporated by reference in their entireties.

The present invention will be further illustrated in the following examples. However, it is to be understood that these examples are for illustrative purposes only, and should not be used to limit the scope of the present invention in any manner.

### EXPERIMENTALS

Materials

Creatininase from Pseudomonas putida was obtained from Roche Diagnostics, Mannheim, Germany. Hydromed D4, Hydromed D640 and Hydromed TP were obtained from Cardiotech International Inc., Wilminton, Mass., USA

General Procedure

Measurement of Apparent Diffusion Coefficient

The diffusion properties can be determined in a diffusion cell, where a value for the apparent diffusion coefficient for a substrate is obtained as the result of the total porosity and the diffusion coefficient of the substrate in water. “Apparent diffusion coefficient” generally refers to the “efficient” diffusion coefficient for the entire membrane area not taking into account the porosity of the membrane.

The diffusion cell (diameter 15 mm, having an O-ring) should be absolutely clean before use. In order to reduce contamination, it is advisable to have the solution with the high substrate concentration in the cell half where the O-ring is arranged. The fluid sample for analysis is loaded into the cell half without the O-ring. A membrane sample approximately ½ cm larger than the opening between the cell halves is cut and is arranged on top of the O-ring. The cell is then closed and tightened. The diffusion cell with the membrane and a magnetic bar (10 mm) is placed on a magnetic stirrer (320±30 r.p.m.). Approximately 30 mL of a substrate solution in cleaning liquid (S4970) and 30 mL of pure cleaning liquid (S4970) are simultaneously loaded into the two half cells of the diffusion cell. After 48 and 72 hours, respectively, the substrate concentration in the pure cleaning liquid is measured by taking out a 1 mL sample with a syringe and filling up with 1 mL of the pure cleaning liquid. The substrate concentration in the samples is measured on an ABL™ 735 Blood Gas Analyzer (Radiometer Medical ApS, Copenhagen, Denmark).

The apparent diffusion coefficient is determined as follows. The flux: in all systems, a passive transport process of a compound will take place if the distribution of the compound in the system does not correspond to the thermodynamic equilibrium distribution of the compound. The flux is defined as the amount of compound which passes through an area unit (area perpendicular to the direction of the transport) per second, and has the unit J=amount·cm⁻²·s⁻¹.

Fick's 1st law is valid for a stationary diffusion, i.e. a linear concentration gradient has been established.

$$J = - D \frac{ⅆ C}{ⅆ x}$$

where D is the diffusion coefficient for the compound, i.e., a value characteristic for the diffusing molecule type under the given conditions (it does not only include the factors determining the rate of transport, such as size and form, but also properties of the surrounding medium like, e.g., viscosity); dC/dx is the slope of the concentration profile at point x (the value dC/dx is also referred to as the concentration gradient in direction x, where the sign character shows the direction in which the concentration increases, i.e., a positive value for dC/dx shows that the concentration increases in the positive direction of the x-axis).
General Sensor Construction (Conventional Sensor Type)

With reference to FIG. 1, the sensor 1 comprises an electrode 2 onto which a membrane ring 3 is attached. The electrode 2 comprises a platinum anode 4 connected with a platinum wire 5 which, through a micro plug 6, is connected with a silver anode contact body 7. The platinum anode 4 and the lower part of the platinum wire 5 are sealed into a glass body 8. Between the glass body 8 and the micro plug 6, the platinum wire 5 is protected with a heat shrink tubing. A tubular silver reference electrode 10 encircles the upper part of the glass body 8 and extends in the length of the electrode 2 to the anode contact body 7 which is fastened inside the reference electrode by means of a fixing body 11 and epoxy 12. The lower part of the glass body 8 is surrounded by an electrode base 13 whereto the membrane ring 3 is attached.

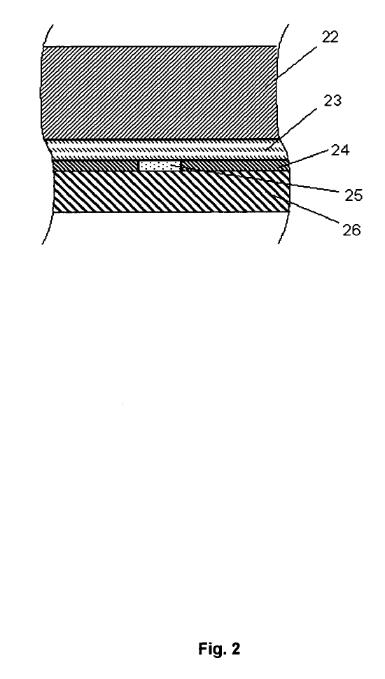

With reference to FIGS. 1 and 2, the upper part of the reference electrode 10 is surrounded by a plug part 14 for mounting the electrode 2 in the corresponding plug of an analysis apparatus (not shown) and for fixing a mantle 15. Gaskets 16 and 17 are placed between the electrode 2 and the mantle 15 in order to ensure that any electrolyte located at the measuring surface of the electrode 2 does not evaporate. The membrane ring 3, which is mounted at one end of the mantle 15, comprises a ring 20. A membrane 21 is stretched over the lower opening of the ring 20. This membrane 21 is shown in detail in FIG. 2 and as described in detail in Example 1.

General Sensor Construction (Thick-Film Sensor Type)

FIG. 3 illustrates an exemplary planar, thick-film sensor construction formed on a dielectric substrate (110) where a working electrode (120) and a reference electrode (130;140) are formed. The electrodes are bordered by a two-layer dielectric encapsulant (150;160 and 151;161). The working electrode is covered by a water-containing spacer layer as disclosed herein (121), an intermediate layer (170), an enzyme layer (180), and a cover membrane (190).

FIG. 3 illustrates an exemplary planar, thick-film sensor construction formed on a dielectric substrate (110) where a working electrode (120) and a reference electrode (130;140) are formed. The electrodes are bordered by a two-layer dielectric encapsulant (150;160 and 151;161). The working electrode is covered by a water-containing porous spacer layer (121), an intermediate layer (170), an enzyme layer (180), and a cover membrane (190) as disclosed herein.

Referring to FIG. 3, an alumina substrate 110 of a thickness of 200 μm is provided at one surface with a circular platinum working electrode 120 of a diameter 1000 μm and a thickness of 10 μm, an annular platinum counter electrode 130 of an outer diameter 3000 μm, an inner diameter 2000 μm and a thickness of 10 μm, covering the angular range 30-330° of the outer periphery of the working electrode, and a circular silver/silver chloride reference electrode 140 of a diameter 50 μm, positioned at the outer periphery of the working electrode at 0° C. All of these three electrode structures are connected to the sensor electronics (not shown) across the alumina substrate 110 via platinum filed through holes (not shown) traversing the substrate. Upon operation, the working electrode 120 is polarised to +675 mV vs. the reference electrode 140.

Further on the alumina substrate 110 are two-layered structures of glass and polymer encapsulant. These two-layered structures include an annular structure 160, 161 of an outer diameter 1800 μm, an inner diameter 1200 μm and a thickness of 50 μm surrounding the working electrode 120 and a structure 150, 151 of a thickness 50 μm surrounding the complete electrode system. Both of these two-layered structures consist of an inner layer 150, 160 facing the alumina substrate 110 of ESL glass 4904 from ESL Europe of the United Kingdom of a thickness of 20 μm, and an outer layer 151, 161 of polymer encapsulant from SenDx Medical Inc. of California, USA as disclosed in international patent application WO97/43634 to SenDx Medical Inc. of California, USA which comprises 28.1% by weight of polyethylmethacrylate (Elvacite, part number 2041, from DuPont), 36.4% by weight of carbitol acetate, 34.3% by weight of silaninized kaolin (part number HF900 from Engelhard), 0.2% by weight of fumed silica and 1.0% by weight of trimethoxysilane.

The water-containing porous spacer layer 121 was formed by dispensing 300 nL of a 7% solution of D4 PUR (Hydromed inc.) in 96% ethanol onto the Pt-working electrode by means of microdispensing.

A circular inner membrane 170 of cellulose acetate and cellulose acetate butyrate of a diameter 1200 μm and a thickness of 1 μm covers the working electrode 120 is prepared on top of the spacer layer 121. It is important that the membrane 170 covers all of the spacer-layer, otherwise the exclusion of interferents will not be complete.

A circular enzyme layer 180 of glucose oxidase crosslinked by glutaric aldehyde of a diameter 1200 μm and a thickness of 2 μm covers the inner membrane 170.

The enzyme layer 180 was prepared by dispensing 0.4 μl of a buffered solution of glucose oxidase crosslinked by glutaric aldehyde on the cellulose acetate membrane 170. The enzyme layer was dried 30 min. at 37° C.

A circular cover membrane layer 190 of PVC/trimethylnonyl-triethylene glycol/diethylene glycol of a diameter 4000 μm and a thickness of 10 μm covers the complete electrode system, centered onto the working electrode 120.

The cover membrane was prepared from 1.35 gram of poly vinyl chloride (Aldrich 34,676-4), 0.0149 gram of trimethylnonyl-triethylene glycol (Tergitol TMN3 from Th. Goldschmidt) and 0.134 gram diethylene glycol which were added to 21.3 gram of tetrahydrofurane and 7.58 gram of cyclohexanone. The mixture was stirred until the PVC was dissolved and a homogenous solution was obtained. 28.5 gram of tetrahydrofurane was added to obtain a 2% solution of a 90/1/9 PVC/surfactant/hydrophilic compound composition. The solution was dispensed on the sensor area to cover all three electrodes and to have an approximately 0.5 mm overlap with the polymer encapsulant 151. The cover membranes were dried for 30 min. at 23±2° C. and for 1½ hour at 40° C.

Approximately 0.3 μL of a 5% solution of a hydrophilic polyurethane (Hydromed D640/Hydromed D4 mixture having a water content of 80%) (see Example 1) in 96% EtOH was dispensed onto the dried outer membrane.

All three layers 170, 180, 190 were dispensed on an x,y,z-table mounted with an automatic dispensing unit (IVEK pump).

### Example 1

### Exemplary Creatine and Creatinine Sensor Constructions

Each of the creatine and the creatinine sensors comprise known amperometric sensors. FIG. 1 shows such a sensor 1 (described above) which is suited for mounting in an apparatus for measuring the concentration of analytes in a biological sample, e.g., an ABL™ 735 Blood Gas Analyzer (Radiometer Medical ApS, Copenhagen, Denmark).

FIG. 2 shows details of the membrane 21 comprising four layers: a noise reducing water-containing spacer layer 22 facing the platinum anode 4 of the electrode 2, an interference limiting membrane layer 23, a gasket 24 encircling an enzyme layer 25, and a diffusion limiting porous polymeric material 26 which has been impregnated with a hydrophilic polyurethane having a water content of around 80%. The coated membrane layer 26 faces the sample to be analysed.

The spacer layer 22 may be a 21±2 μm track-edged membrane of polyethylene terephthalate (PETP) (pore diameter approximately 1.3-1.5 μm; pore density: 2.2·10⁷ pores/cm²). The interference limiting membrane layer 23 may be a 6±2 μm porous membrane of cellulose acetate (CA).

The gasket 24 may be a 30±5 μm double-sided adhesive disc having a center hole with a diameter of 1500 μm. The adhesive of the gasket 24 adheres to the interference limiting layer 23 and the diffusion limiting layer 26 to an extent that the enzymes are prevented from leaking out between the layers.

The enzyme layer 25 of the creatine sensor is typically an approximately 20 μm layer of creatinase and sarcosine oxidase crosslinked to glutaraldehyde mixed with suitable additives, such as buffer. The enzyme layer 25 of the creatinine sensor is typically an approximately 20 μm layer of creatininase, creatinase and sarcosine oxidase crosslinked to glutaraldehyde mixed with suitable additives, such as buffer.

The diffusion limiting porous polymeric material 26 may be an approximately 12 μm layer of polyethylenetherephthalate (PETP) (pore diameter approximately 0.1 μm; pore density: 3·10⁷ pores/cm²) which has been impregnated with a hydrophilic polyurethane (Hydromed D640/Hydromed D4 mixture having a water content of 80%) (see Example 1).

In the creatinine sensor, both creatine and creatinine are converted into hydrogen peroxide. In the creatine sensor, only creatine is converted into hydrogen peroxide.

At the amperometric electrode, hydrogen peroxide is oxidized anodically at +675 mV against Ag/AgCl. The resulting current flow is proportional to the creatinine/creatine concentration in the sample.

The concentration of creatinine is determined from the difference between the creatinine sensor signal (representing creatine+creatinine) and the creatine sensor signal (representing creatine).

### Example 2

### Relative Diffusion Coefficients of Imidazole/H-Imidazole⁺ Through a Cellulose Acetate Membrane

The relative diffusion coefficient of imidazole/H-imidazole⁺ through the CA-membrane has been measured by analysing the resulting pH in an unbuffered solution in contact with a solution buffered by imidazole through a CA membrane. Both samples were 30 mL and the CA membrane contacting both solutions was 10 mm in diameter (see “Measurement of Apparent Diffusion Coefficient” above). The solution was 140 mM NaCl, 4 mM KCl, 1 mM CaCl₂ and 110 mM imidazole, pH was adjusted to 7.40 at 25° C. After 20 hours, the pH of the unbuffered solution had risen to 8.86 at 25° C., while the pH of the other solution was unchanged. Thus, the neutral species of imidazole (the basic moiety of the imidazole couple) is able to diffuse at least 30 times faster than the charged species, mirroring the significant changes in pH which may be observed at the electrode surface during exposure to calibration solutions and samples, respectively.

### Example 3

### Influence of a Water-Containing Spacer Layer on Creatinine Sensor Measurements

Two similar blood analysers of the type ABL™ 735 from Radiometer Medical ApS, Denmark, were modified to accommodate the dual sensor system described in Example 1. With one blood analyzer, five creatinine sensors (three-enzyme sensors) and five creatine sensors (two-enzyme sensors) all equipped with a spacer layer according to Example 1 were arranged. Similarly, with a second blood analyzer, five creatinine sensors (three-enzyme sensors) and five creatine sensors (two-enzyme sensors) were arranged according to Example 1, but without the spacer layer.

The calibration solutions were prepared by dissolving 200 μM creatinine in the Radiometer Calibration Solution 1 S1720 and 200 μM creatine in the Radiometer Calibration Solution 2 S1730. These solutions were repeatedly introduced into the apparatus one after the other and sensor responses were obtained.

Following such calibration, the sensors were subjected to the following seven creatinine-free and creatine-free liquids to measure the “false” creatine response thereof (see Table A).

TABLE A
Constitution of seven zero-current liquids.

| | Liquid nr. | | | | | | |
|---|---|---|---|---|---|---|---|
| | 1 | 2 | 3 | 4 | 5 | 6 | 7 |
| NaCl | 120 | 40 | 120 | 120 | 120 | 40 | 40 |
| KCl | 4 | 4 | 4 | 12 | 4 | 4 | 4 |
| CaCl₂ | 2 | 2 | 2 | 2 | 2 | 2 | 2 |
| NaHCO₃ | 25 | 25 | 25 | 25 | 10 | 10 | 50 |
| pH | 7.2 | 7.2 | 6.5 | 7.2 | 7.2 | 7.2 | 7.2 |

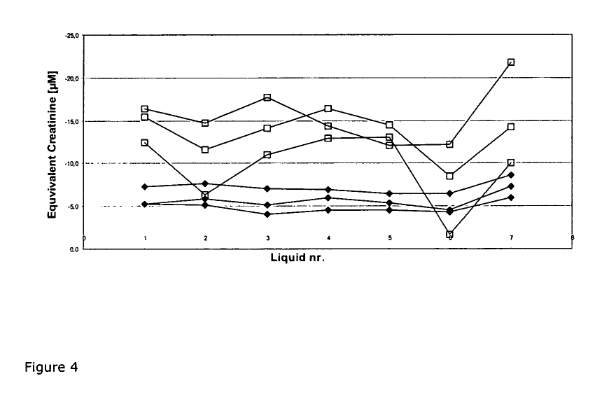

The sensor signal is calculated as the difference in signal immediately before the sensor is subjected to the sample and the signal 28 sec after sample contact. Likewise, the sensor sensitivity is calculated from the calibration solutions. The sensor signal on the 7 liquids is then converted to a corresponding “false” creatine concentration to compare both 2enz. and 3enz. sensors using the sensor sensitivity (see Tables B and C and FIG. 4). It is noted that the expected value is “0” (zero).

TABLE B
Creatine signals (in μM) from the seven zero current liquids on sensors with a
water-containing spacer layer.

| | Liquid 1 | Liquid 2 | Liquid 3 | Liquid 4 | Liquid 5 | Liquid 6 | Liquid 7 |
|---|---|---|---|---|---|---|---|
| 2enz | −7.99 | −5.98 | −5.16 | −5.13 | −1.46 | −3.75 | −6.07 |
| 2enz | −4.12 | −4.21 | −3.29 | −4.99 | −2.63 | −2.80 | −6.15 |
| 2enz | −6.29 | −4.52 | −3.17 | −4.45 | −2.60 | −2.48 | −5.84 |
| 2enz | −7.73 | −6.18 | −5.03 | −6.11 | −4.20 | −4.41 | −7.28 |
| 2enz | −4.85 | −4.19 | −3.33 | −4.43 | −2.06 | −2.28 | −4.78 |
| 3enz | −6.56 | −5.68 | −4.17 | −6.08 | −3.19 | −3.90 | −7.47 |
| 3enz | −7.02 | −4.44 | −4.43 | −7.27 | −3.66 | −2.09 | −7.17 |
| 3enz | −5.92 | −4.41 | −2.85 | −6.30 | −2.62 | −2.48 | −6.42 |
| 3enz | −6.16 | −5.58 | −4.11 | −5.89 | −2.70 | −3.05 | −7.24 |
| 3enz | −6.38 | −5.46 | −3.97 | −5.40 | −3.15 | −2.91 | −7.09 |
| Average 2enz | −6.20 | −5.02 | −4.00 | −5.02 | −2.59 | −3.15 | −6.02 |
| Average 3enz | −6.41 | −5.11 | −3.91 | −6.19 | −3.07 | −2.89 | −7.08 |
| Difference | −0.21 | −0.10 | 0.09 | −1.17 | −0.48 | 0.26 | −1.05 |
| Std. dev. | 1.08 | 0.76 | 0.70 | 0.93 | 0.65 | 0.76 | 0.89 |

TABLE C
Creatine signals (in μM) from the seven zero current liquids on sensors without
water-containing spacer layer.

| | Liquid 1 | Liquid 2 | Liquid 3 | Liquid 4 | Liquid 5 | Liquid 6 | Liquid 7 |
|---|---|---|---|---|---|---|---|
| 2enz | −11.52 | −7.15 | −10.36 | −10.18 | −9.10 | −4.64 | −10.08 |
| 2enz | −7.66 | −8.38 | −7.05 | −8.61 | −3.95 | −4.65 | −12.42 |
| 2enz | −12.18 | −11.53 | −11.19 | −10.96 | −9.00 | −9.07 | −14.79 |
| 2enz | −14.25 | −12.96 | −11.64 | −12.00 | −9.77 | −9.90 | −15.99 |
| 2enz | −12.26 | −8.28 | −9.88 | −10.70 | −8.29 | −4.69 | −11.64 |
| 3enz | −12.27 | −11.37 | −11.57 | −12.88 | −9.27 | −7.25 | −16.86 |
| 3enz | −13.93 | −13.29 | −11.87 | −13.34 | −9.24 | −9.62 | −18.21 |
| 3enz | −16.45 | −12.84 | −11.96 | −12.34 | −10.00 | −8.49 | −16.53 |
| 3enz | −10.71 | −3.80 | −7.82 | −13.28 | −6.68 | 1.16 | −11.54 |
| Average 2enz | −11.57 | −9.66 | −10.02 | −10.49 | −8.02 | −6.59 | −12.98 |
| Average 3enz | −13.34 | −10.33 | −10.80 | −12.96 | −8.80 | −6.05 | −15.78 |
| Difference | −1.77 | −0.67 | −0.78 | −2.47 | −0.78 | 0.54 | −2.80 |
| St. dev. | 2.46 | 3.24 | 1.81 | 1.59 | 1.92 | 3.55 | 2.87 |

As can be seen from the Tables B and C, sensors equipped with a spacer layer show less difference (i.e., the difference between “false” creatine signals from 2enz sensors and 3enz sensors, respectively) and less standard deviation compared to sensors with no water-containing spacer layer.

A similar series of experiments was conducted with 3enz sensors with and without the water-containing spacer layer. The results are—in this instance—converted to “false” creatinine signals. The results are shown in FIG. 4 (black diamonds represent sensors with a spacer layer and open squares represent sensors without a spacer layer). The sensor-to-sensor variation is reduced with the introduction of a spacer layer.

### Example 4

### Influence of a Water-Containing Spacer Layer on Creatine/Creatinine Sensor Measurements on Whole Blood

A whole blood sample from a healthy individual was measured on the above described sensors from Example 1. The creatinine concentration of the patient blood samples was calculated using all combinations of five creatine sensors and four creatinine sensors from each of the above two branches from Example 3 (with and without a spacer layer) (see Tables D and E).

TABLE D
Creatinine signals (in μM) for sensors with a water-containing
sapcer layer. Measurements on a real blood sample.

| | Creatinine 1 | Creatinine 2 | Creatinine 3 | Creatinine 4 | | |
|---|---|---|---|---|---|---|
| Creatine 1 | 88.75 | 84.91 | 83.97 | 83.76 | Max | 88.86 |
| Creatine 2 | 86.51 | 82.63 | 81.74 | 81.59 | Min | 81.32 |
| Creatine 3 | 86.24 | 82.35 | 81.46 | 81.32 | Span | 7.54 |
| Creatine 4 | 88.74 | 84.90 | 83.96 | 83.75 | Mean | 84.42 |
| Creatine 5 | 88.86 | 85.02 | 84.08 | 83.86 | Std. Dev. | 2.30 |

TABLE E
Creatinine signals (in μM) for sensors without a water-containing
spacer layer. Measurements on a real blood sample.

| | Creatinine 1 | Creatinine 2 | Creatinine 3 | Creatinine 4 | | |
|---|---|---|---|---|---|---|
| Creatine 1 | 71.69 | 70.66 | 81.75 | 77.38 | Max | 86.39 |
| Creatine 2 | 76.20 | 75.08 | 86.39 | 82.16 | Min | 70.66 |
| Creatine 3 | 71.98 | 70.94 | 82.0.5 | 77.69 | Span | 15.73 |
| Creatine 4 | 75.75 | 74.64 | 85.93 | 81.68 | Mean | 77.32 |
| Creatine 5 | 72.41 | 71.37 | 82.49 | 78.14 | Std. Dev. | 5.07 |

As can be seen from the Tables D and E the span and standard deviation is lower for sensors with a spacer layer.

### Example 5

### Sensitivity to H₂O₂

Three simplified planar sensors only comprising a platinum electrode, a water-containing spacer layer and an interference reducing layer were constructed essentially as described above under “General sensor construction (thick-film sensor type)”.

The water-containing porous spacer layer was formed by dispensing 300 nL of a 7% solution of D4 PUR (Hydromed inc.) in 96% ethanol onto the Pt-working electrode by means of microdispensing, and the inner membrane of cellulose acetate and cellulose acetate butyrate of a thickness of 1 μm was prepared on top of the spacer layer.

Two reference planar sensors were prepared as described before, but without the spacer layer and an inner membrane thickness of 2 μm.

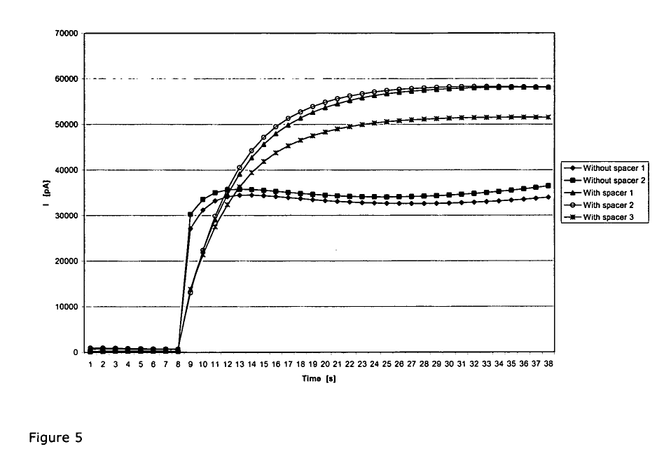

A series of measurement were conducted using the five sensors and using a 1 mM H₂O₂ solution as the test sample. The H₂O₂ solution was used to mimic a glucose oxidase layer exposed to glucose. The results are illustrated in FIG. 5.

A higher sensitivity is observed for sensors having a spacer layer (approximately 55 nA) than for sensors not having a spacer layer (approximately 35 nA). This is believed to be the result of an improved diffusion of H₂O₂ to the platinum electrode, because the spacer allows for a high diffusion rate and because the cellulose acetate inner membrane may be dispensed in a very thin layer when the spacer layer is present.

Unless otherwise defined, all technical and scientific terms herein have the same meaning as commonly understood by one of ordinary skill in the art to which this invention belongs. Although any methods and materials, similar or equivalent to those described herein, can be used in the practice or testing of the present invention, the preferred methods are described herein. All publications, patents and patent applications cited herein are incorporated by reference for the purpose of disclosing and describing specific aspects of the invention for which the publication, patent or patent application is cited.

1. An amperometric enzyme sensor for determining the concentration of an analyte in a fluid sample, comprising
an electrode,
a water-containing spacer layer in contact with said electrode,
at least one intermediate layer, the innermost of said at least one intermediate layer being in contact with said spacer layer, and
at least one enzyme layer, the innermost of said at least one enzyme layer being in contact with the outermost of said intermediate layer(s).

2. The enzyme sensor according to claim 1, wherein the spacer layer has a porosity of in the range of about 0.0005 to about 2% (vol/vol) for track-etched materials and in the range of about 1 to about 90% for solvent-cast materials.

3. The enzyme sensor according to claim 1, wherein the spacer layer comprises water and solid matter, and the weight ratio between the water and the solid matter is in the range of from about 10:1 to about 1:10.

4. The enzyme sensor according to claim 1, wherein the spacer layer has a thickness of in the range of about 0.2 to about 20 μm.

5. The enzyme sensor according to claim 1, wherein the water-containing spacer layer further comprises one or more components selected from buffers, electrolyte salts and cation-exchange materials.

6. The enzyme sensor according to claim 1, wherein the sensor is a conventional sensor and the spacer layer comprises solid matter, the solid matter essentially consisting of a porous polymeric matrix selected from polyethylene terephthalate (PETP), polyvinyl chloride, and polycarbonate.

7. The enzyme sensor according to claim 6, wherein the porous polymeric matrix is a polyethylene terephthalate (PETP) material.

8. The enzyme sensor according to claim 1, wherein the porous polymeric matrix of the spacer layer is a track-etched membrane.

9. The enzyme sensor according to claim 1, wherein the sensor is a planar sensor type and the spacer layer comprises solid matter, the solid matter essentially consisting of a porous polymeric matrix selected from hydrophilic polyurethanes, hydrophilic poly(meth)acrylates, poly(vinyl pyrrolidone), polyurethanes, Nafion™-polymers, electropolymerised polymers, and SPEES-PES.

10. The enzyme sensor according to claim 1, wherein the porous polymeric matrix of the spacer layer is a solvent-cast layer.

11. The enzyme sensor according to claim 1, comprising
an electrode,
a water-containing spacer layer in contact with said electrode,
an intermediate layer in contact with said spacer layer, and
an enzyme layer in contact with said intermediate layer.

12. The enzyme sensor according to claim 1, wherein the enzyme layer comprises creatinase and/or creatininase.

13. The enzyme sensor according to claim 1, further comprising a cover membrane for said at least one enzyme layer.

14. The enzyme sensor according to claim 13, wherein the cover membrane comprises at least one porous polymeric material, wherein the outer surface and pore mouths of at least one face of the at least one porous polymeric material are covered by a hydrophilic polymer selected from hydrophilic polyurethanes and hydrophilic poly(meth)acrylates.

15. An amperometric enzyme sensor for determining the concentration of creatine in a fluid sample, comprising
a metal electrode,
a water-containing spacer layer in contact with said metal electrode,
an interference limiting layer in contact with said spacer layer,
an enzyme layer comprising sarcosine oxidase and creatinase in contact with said interference limiting layer, and
a cover membrane layer for said enzyme layer, wherein said cover membrane layer comprises a porous polyethylene terephthalate material, and wherein the outer surface and pore mouths of at least one face of the porous polyethylene terephthalate material are covered by a hydrophilic polyurethane comprising backbone segments of polyethylene glycol in a weight ratio of polyethylene glycol segments of at least about 5% (w/w) and/or have a water content when wetted of at least about 25% (w/w).

16. The sensor according to claim 15, wherein the porous polyethylene terephthalate material is a track-etched material.

17. An amperometric enzyme sensor for determining the concentration of creatinine in a fluid sample, comprising
a metal electrode,
a water-containing spacer layer in contact with said metal electrode,
an interference limiting layer in contact with said spacer layer,
an enzyme layer comprising sarcosine oxidase, creatininase and creatinase in contact with said interference limiting layer, and
a cover membrane layer for said enzyme layer, wherein said cover membrane layer comprises a porous polyethylene terephthalate material, and wherein the outer surface and pore mouths of at least one face of the porous polyethylene terephthalate material are covered by a hydrophilic polyurethane comprising backbone segments of polyethylene glycol in a weight ratio of polyethylene glycol segments of at least about 5% (w/w) and/or have a water content when wetted of at least about 25% (w/w).

18. The sensor according to claim 17, wherein the porous polyethylene terephthalate material is a track-etched material.

19. An apparatus for determining the concentration of an analyte in a fluid sample, comprising one or more enzyme sensors as defined in any one of the claims 1, 15 and 17.

20. A method of determining the concentration of an analyte in a fluid sample, comprising the steps of
contacting the fluid sample with an enzyme sensor according to any one of the claims 1, 15 and 17, and
conducting at least one measurement involving the electrode of the enzyme sensor.
